(19) United States
(12) Patent Application Publication
(10) Pub. No.: US 2017/0202751 A1
(43) Pub. Date: Jul. 20, 2017

(54) Dental Etchant Compositions Comprising One Or More Dentin Collagen Cross-Linking Agents, Methods Of Producing The Same, And Methods Of Use

(71) Applicant: The Curators of the University of Missouri, Columbia, MO (US)

(72) Inventors: YONG WANG, Overland Park, KS (US); YI LIU, Overland Park, KS (US)

(21) Appl. No.: 15/127,652

(22) PCT Filed: Nov. 26, 2014
(86) PCT No.: PCT/US14/67557

§ 371 Date: Sep. 20, 2016

Related U.S. Application Data
(60) Provisional application No. 61/967,559, filed on Mar. 20, 2014.

(51) Int. Cl. A61K 6/00; A61C 19/06; A61C 5/00

(52) U.S. Cl. CPC A61K 6/0029; A61C 5/00; A61C 19/063; A61C 2201/002

(57) ABSTRACT

Dental etchant compositions, processes for making the same, and methods for using the dental etchant compositions are disclosed. The dental etchant compositions include at least one acid and one or more dentin collagen cross-linking agents. The methods for using the dental etchant compositions may include contacting a mammal's tooth with a dental etchant composition that includes one or more acids and one or more dentin collagen cross-linking agents.

### FIELD OF THE INVENTION

The disclosure herein relates to compositions for use in dental restorations. Particularly, the disclosure herein relates to dental etchant compositions comprising one or more dentin collagen cross-linking agents and methods of producing the same. The disclosure herein also relates to methods for using dental etchant compositions comprising one or more dentin collagen cross-linking agents.

### SUMMARY OF THE INVENTION

This Summary is provided to introduce a selection of concepts in a simplified form that are further described below in the Detailed Description. This Summary is not intended to identify key features or essential elements of the claimed subject matter, nor is it intended to be used as an aid in determining the scope of the claimed subject matter.

Certain dental restorations may utilize a composite or an amalgam material. For various reasons, such as cosmetic preference, one may choose to utilize a composite material in a dental restoration. However, certain current composite tooth restorations exhibit a shortened life-span compared to a tooth restoration performed with a traditional amalgam filling. In certain situations, this shortened life-span may be due to a breakdown of at least a portion of the dentin collagen that interfaces with the composite material. Therefore, there is a need for compositions that can be used in a dental restoration process, such as a composite or ceramic dental restoration process, that increase the stability of dentin collagen.

Accordingly, aspects herein describe dental etchant compositions comprising one or more dentin collagen cross-linking agents and processes for making the same. In addition, aspects herein describe processes for performing a dental restoration using dental etchant compositions comprising one or more dentin collagen cross-linking agents.

In one aspect, a dental etchant composition is disclosed. The dental etchant composition includes at least one acid and one or more dentin collagen cross-linking agents.

In another aspect, a process for the production of a dental etchant composition is disclosed. The process includes combining at least one acid with one or more dentin collagen cross-linking agents to form a dental etchant composition.

In yet another aspect, a method for performing a dental restoration on a mammal's tooth is disclosed. The method includes contacting at least a portion of a mammal's tooth with a dental etchant composition. The dental etchant composition includes one or more acids and one or more dentin collagen cross-linking agents. The method further includes removing at least a portion of the dental etchant composition from the mammal's tooth.

### BRIEF DESCRIPTION OF THE DRAWING

Aspects herein are described in detail with reference to the attached drawing figures, wherein:

FIG. 1 depicts the FTIR spectra of dentin films with untreated collagen, or of dentin films treated with various GSE-containing etchant formulations or GSE powder, as described in Example 1;

FIG. 2 depicts the standard curve of the MALDI-based collagenase digestion assay described in Example 2;

FIG. 3 is a bar graph depicting the percentage of the dentin film degraded by one hour of bacterial collagenase digestion using the MALDI-based collagenase digestion assay described in Example 2;

FIG. 4A is an optical image of the fractured cross-section of a representative dentin film specimen etched by GSE10 for 30 seconds as described in Example 3;

FIG. 4B depicts the Raman spectra collected from the specimen shown in FIG. 4A;

FIG. 4C depicts the 30 second etching depth of the GSE20, GSE10, and GSE5 treated dentin slabs based on Raman spectra as described in Example 3;

FIG. 5A-5H depict SEM images of a cross-section of fractured dentin slabs treated with various etchant formulations, with and without collagenase digestion, as described in Example 4; and

FIGS. 6A-6H depict TEM images of dentin slab specimens etched with GSE20, GSE10, and GSE5 etchant formulations, with and without collagenase digestion, as described in Example 4.

### DETAILED DESCRIPTION OF THE INVENTION

Aspects herein describe dental etchant compositions that include an acid and one or more dentin collagen cross-linking agents. In one or more aspects, the acid can include a mineral acid, such as phosphoric acid. In certain aspects, the acid can include an organic acid, such as maleic acid and/or citric acid. In one aspect, the acid can be selected from the group consisting of phosphoric acid, maleic acid, citric acid, and combinations thereof. In various aspects, the acid present in the dental etchant compositions can function as a chemical etchant that can demineralize at least a portion of the enamel of a mammal's tooth.

In various aspects, the acid can be present in the dental etchant compositions in an amount of at least about 0.01, 0.05, 0.1, 0.5, 1, or 5 weight (“wt.”) %, and/or not more than about 50, 40, 30, or 20 wt. %. As used herein, wt. % refers to the weight percent of a component based on the total weight of the dental etchant composition. In certain aspects, the acid in the dental etchant compositions can include phosphoric acid in an amount of at least about 1 wt. % and/or not more than about 20 wt. %. In another, the acid in the dental etchant compositions can include phosphoric acid in an amount of at least about 1 wt. % and/or not more than about 40 wt. %.

In various aspects, the one or more dentin collagen cross-linking agents present in the dental etchant compositions can be any compound that is capable of interacting with dentin collagen in an acidic environment in such a manner so as to stabilize the dentin collagen (e.g., by protecting it from enzymatic degradation). In such aspects, at least a portion of the dentin collagen cross-linking agent may interact with one or more residues of the dentin collagen, such as proline and/or hydroxyproline, in such a manner so as to stabilize the triple helical structure of the dentin collagen. In such aspects, at least a portion of the one or more dentin collagen cross-linking agents may be a non-covalent dentin collagen cross-linking agent.

In certain aspects, one or more dentin collagen cross-linking agents present in the dental etchant compositions may function as a non-covalent cross-linking agent that may couple together one or more collagen fibers in a low pH environment, such as a pH of less than about 4, 3, 2, or 1. In the same or alternative aspects, the one or more dentin collagen cross-linking agents present in the dental etchant compositions may function as a collagen stabilizing agent such that at least a portion of dentin collagen can be protected from collagenase digestion, compared to the level of collagenase digestion performed in the absence of one or more dentin collagen cross-linking agents.

In one or more aspects, the dentin collagen cross-linking agent can comprise, consist essentially of, or consist of one or more polyphenols. In aspects, the one or more polyphenols can include naturally occurring polyphenols. In one aspect, the one or more polyphenols can consist, or consist essentially of, naturally occurring polyphenols. In the same or alternative aspects, the dental etchant compositions may contain only naturally occurring polyphenols. As used herein, naturally occurring polyphenols refers to polyphenols that were extracted and/or isolated from a natural source, such as a plant. In certain aspects, the plant extract may include extracts of grape seeds, apples, grape skin, cinnamon, maritime pine bark, aronia fruit, cocoa beans, bilberry, cranberry, black currant, green tea, black tea, oak trees, and/or a [symbol] a [symbol] oil from the fruit of the a [symbol] a [symbol] palm.

In certain aspects, the one or more dentin collagen cross-linking agents can include one or more proanthocyanidins. In various aspects, the proanthocyanidins and/or the dentin collagen cross-linking agents may be a component of one or more plant extracts and/or plant seed extracts that are present in the dental etchant compositions. The plant extract and/or plant seed extract may include any plant extract and/or a plant seed extract known to one skilled in the art that includes proanthocyanidins or a dentin collagen cross-linking agent. For example, in certain aspects, the plant extract and/or plant seed extract may include extracts of grape seeds, apples, grape skin, cinnamon, maritime pine bark, aronia fruit, cocoa beans, bilberry, cranberry, black currant, green tea, black tea, oak trees, and/or a [symbol] a [symbol] oil from the fruit of the açaí palm.

In the same or alternative aspects, the dental etchant compositions can include one or more proanthocyanidin-mimicking compounds. As used herein, proanthocyanidin-mimicking compounds refers to any compound, or portion of a compound, having a substantially similar structure to at least a portion of a proanthocyanidin compound, such as, for example, compounds that include a 1,2-benzenediol moiety, a 1,3-benzenediol moiety, and/or a trihydroxybenzoic acid. In aspects, other compounds having a substantially similar structure to one or more proanthocyanidins can include one or more flavanols (derivatives of flavans). In various aspects, other compounds having a substantially similar structure to one or more proanthocyanidins can include monomeric, dimeric, trimeric, and/or oligomeric compounds or polyphenols having one or more units that exhibit a substantially similar structure to catechin, epicatechin gallate, epigallocatechin, epigallocatechin gallate, and/or isomers thereof.

In certain aspects, the one or more dentin collagen cross-linking agents can be present in the dental etchant compositions in an amount of at least about 0.01, 0.05, 0.1, or 0.5 wt. %, and/or not more than about 20, 15, 10, or 5 wt. %. In one or more aspects, when at least a portion of the one or more dentin collagen cross-linking agents is present in the dental etchant compositions via a plant extract and/or a plant seed extract, such extract may be present in an amount of at least about 0.01, 0.05, 0.1, or 0.5 wt. %, and/or not more than about 20, 15, 10, or 5 wt. %.

In certain aspects, the ratio of acid to dentin collagen cross-linking agents present in the dental etchant compositions can be at least about 50:1, 40:1, 30:1, 20:1, 10:1, 7.5:1, 5:1, or 2.5:1. In various aspects, the ratio of acid to dentin collagen cross-linking agents present in the dental etchant compositions can be no more than about 50:1, 40:1, 30:1, 20:1, 10:1, 7.5:1, 5:1, or 2.5:1. In one or more aspects, the dental etchant compositions may have a pH of at least about 0.01, 0.05, 0.1, 0.15, or 0.2, and/or less than about 7, 6, 5, 4, 3, 2, or 1.

In certain aspects, the acid in the dental etchant compositions may be damaging to the tissues adjacent to the tooth. Accordingly, in such aspects, in an attempt to minimize the flow of the dental etchant compositions from the tooth surface to the surrounding tissues it may be desirable to increase the viscosity of the dental etchant compositions, e.g., by formulating the dental etchant composition as a gel or a paste. In various aspects, the dental etchant compositions can have a viscosity of at least about 25, 50, 75, or 100 centipoise (cps), and/or not more than about 15,000, 10,000, 5,000, 4000, 3000, 2500, 2000, 1000, or 500 cps. In one or more aspects, the dental etchant compositions may include one or more rheology modifiers. Any rheology modifiers known to one skilled in the art can be used as long as such a rheology modifier does not interfere with the function of the dentin collagen cross-linking agents and/or the acid. For example, in such aspects, silica, polyvinyl alcohol, or other rheology modifiers known in the art may be utilized in the dental etchant compositions. In one aspect, the rheology modifiers may be selected from the group consisting of: silica, polyvinyl alcohol, and a combination thereof. One skilled in the art understands how to use such rheology modifiers, and therefore, they are not further discussed herein.

In certain aspects, it may be desirable for the dental etchant compositions to be colored different than a tooth and/or the surrounding tissue so that one applying the etchant composition, e.g., a dental technician, dental hygienist, and/or a dentist, can readily see the area that the dental etchant composition was applied to. In such aspects, the dental etchant compositions may include a dye or other colorant. Any dye or colorant known to one skilled in the art may be used in the dental etchant compositions as long as such a dye or colorant does not affect the function of the acid and/or the dentin collagen cross-linking agents. For example, in various aspects, one or more Federal Food, Drug, and Cosmetic Act (FD&C) approved dyes may be present in the dental etchant compositions disclosed herein. One skilled in the art understands how to use such dyes or other colorants in the dental etchant compositions disclosed herein, and therefore, they are not further discussed.

In various aspects, one or more components of the dental etchant compositions disclosed herein may be colored differently than a tooth and/or the surrounding tissue. For example, in certain aspects, one or more dentin collagen cross-linking agents may be colored differently than a tooth and/or the surrounding tissue, such as when the dentin collagen cross-linking agents are present in a plant or plant seed extract. In such aspects, the dental etchant compositions disclosed herein may not include an additional dye or colorant, such as one or more FD&C approved dyes.

In certain aspects, the dental etchant compositions may include one or more solvents. In such aspects, the solvent can be any aqueous solvent, such as water, as long as the solvent would not affect the function of the acid and/or the dentin collagen cross-linking agents. In certain aspects, the dental etchant compositions can include an aqueous and/or an organic solvent, such as ethanol.

In various aspects, the dental etchant compositions may consist essentially of one or more acids and one or more dentin collagen cross-linking agents. In one or more aspects, the dental etchant compositions may consist essentially of phosphoric acid and one or more polyphenols. In various aspects, the dental etchant compositions may consist essentially of phosphoric acid and one or more proanthocyanidins. In certain aspects, the dental etchant compositions may consist essentially of one or more acids, one or more dentin collagen cross-linking agents, and one or more rheology modifiers. In various aspects, the dental etchant compositions may consist essentially of one or more acids, one or more polyphenols, and one or more rheology modifiers. In one or more aspects, the dental etchant compositions may consist essentially of phosphoric acid, proanthocyanidins, and one or more rheology modifiers.

As discussed above, aspects disclosed herein described a process for the production of dental etchant compositions that may include combining one or more acids with one or more dentin collagen cross-linking agents. In such aspects, the acid may include any or all of the properties of the acids discussed above. For example, in one or more aspects, the acid can include a mineral acid, such as phosphoric acid, and/or an organic acid, such as maleic acid and/or citric acid. In various aspects, the one or more dentin collagen cross-linking agents can include any or all of the properties of the dentin collagen cross-linking agents discussed above. For example, in such aspects, the one or more dentin collagen cross-linking agents can include one or more proanthocyanidins and/or one or more pro anthocyanidin-mimicking compounds.

In one or more aspects, the one or more acids and one or more dentin collagen cross-linking agents can be combined in any manner known to one skilled in the art and may depend upon the nature of the chosen acid and dentin collagen cross-linking agents. For example, in one aspect, the one or more acids may include one or more mineral acids in an aqueous solution, to which one or more solid or liquid sources of dentin collagen cross-linking agents may be added. A solid source of dentin collagen cross-linking agents may include a ground-up powdered plant seed extract, such as grape seed extract. Further, in such aspects, one or more rheology modifiers and/or dyes or other colorants may be added to the acid and dentin collagen cross-linking agent mixture. One skilled in the art will appreciate that the components of the dental etchant compositions disclosed herein may be added in any order known to such a skilled artisan.

In various aspects, the process for producing a dental etchant composition may include increasing the viscosity of a mixture comprising one or more acids and one or more dentin collagen cross-linking agents, e.g., by adding one or more rheology modifiers. In the same or alternative aspects, the process for producing a dental etchant composition may include formulating a mixture comprising one or more acids and one or more dentin collagen cross-linking agents into a gel or paste, using techniques known to one skilled in the art.

Aspects herein also describe processes for performing a dental restoration using dental etchant compositions that comprise one or more acids and one or more dentin collagen cross-linking agents. In certain aspects, the dental restoration can be a restoration utilizing a composite and/or a ceramic material, or any other material used to perform a dental restoration. In aspects, the dental etchant compositions can include any of the properties and/or parameters of the dental etchant compositions discussed above. For example, the dental etchant compositions utilized in the processes for performing a composite restoration disclosed herein may exhibit the type and concentrations of dentin collagen cross-linking agents discussed above, the type and concentration of acids discussed above, the compositional pH values discussed above, and/or the compositional viscosity discussed above.

In one or more aspects, a process for performing a dental restoration can include contacting the dental etchant composition to at least a portion of a mammal's tooth, e.g., a human's tooth. In aspects, the dental etchant composition can be applied to a mammal's tooth in any manner known to one skilled in the art, such as via a syringe, a brush, and/or cotton pellets. In one or more aspects, the dental etchant composition may contact the tooth for at least about 1, 5, 10, 15, or 20 seconds, and/or not more than about 60, 45, or 30 seconds.

In various aspects, after contacting the dental etchant composition to the tooth, the dental etchant composition may be removed using any techniques known to one skilled in the art, such as by rinsing the dental etchant composition off the tooth. In one or more aspects, the process for performing a dental restoration can further include providing an adhesive resin to the tooth. Applying an adhesive resin to a tooth can be done using any techniques known to one skilled in the art and is therefore not further described herein.

### EXAMPLES

The concepts described herein will be further described in the following examples, which do not limit the scope of the claims.

### Example 1: Demineralized Dentin Interacts with and Immobilizes Proanthocyanidins in the Presence of Phosphoric Acid

Three Grape seed extract (GSE)-containing etchant formulations (GSE20, GSE10, and GSE5) were prepared by mixing GSE-powder, ethanol, deionized water (DW) and 85% phosphoric acid to final concentrations (weight percentage (wt. %) with respect to total mass) of 2% GSE, 20% ethanol and 20%, 10%, and 5% phosphoric acid, respectively. Chemicals were purchased from Sigma-Aldrich (St. Louis, Mo.) unless stated otherwise and the GSE was MegaNatural® Gold GSE (Lot#: 05592502-01) by Polyphenolics, Madera, Calif. Three glutaraldehyde (GA)-containing etchant formulations (GA20, GA10, and GA5) were similarly made except that the GA was present, instead of the GSE, in an amount of 2.5 wt. %. A collagenase (Type-I, from Clostridium histolyticum, ≧125 U/mg)-containing solution was made at 0.1% (w/v) in TESCA buffer (50-mM N-tris(hydroxymethyl)methyl-2-aminoethanesulfonic acid, 0.36-mM CaCl₂, pH=7.4).

Sixteen non-carious human third molars were collected from young adults with patients' informed consent under a protocol approved by University of Missouri-Kansas City Adult Health Sciences IRB. The extracted teeth were stored at 4° C. in 0.96% (w/v) phosphate buffered saline containing 0.002% sodium azide. Six randomly-selected teeth were processed into dentin films, and the rest into dentin slabs as described below. For each of the 6 teeth, the occlusal portion of crown and side walls of enamel were removed by a water-cooled low-speed diamond saw (Buehler, Lake Bluff, Ill.). The resultant dentin block was sectioned in the mesial-distal direction, with a tungsten carbide knife mounted on a SM2500S microtome (Leica, Deerfield, Ill.) into 6-μm-thick films. Fifty films were obtained from each tooth, resulting in a pool of 300 films with uniform size of approximately 5 mm×5 mm.

From this pool of 300 films, 30 films were randomly selected and assigned to 6 groups (n=5) and treated by the GSE-containing etchant formulations or the GA-containing etchant formulations as follows. Each dentin film was completely demineralized with 35% phosphoric acid for 15 seconds, rinsed in DW, and spread on a plastic cover slip. After blotting away excessive water, a small drop of selected GA-containing or GSE-containing etchant formulation was applied to cover the entire film. After 30 seconds, the film was rinsed and immersed in copious DW for 30 minutes to thoroughly remove residual treatment solution.

After overnight air-drying, FTIR spectra of the films were collected at 4-cm⁻¹ resolution and 128 scans using an FTIR spectrometer equipped with attenuated total reflectance (ATR) attachment (Spectrum One, Perkin-Elmer, Waltham, Mass.). Band area calculations were performed with Spectrum software (Perkin-Elmer) following two-point baseline correction.

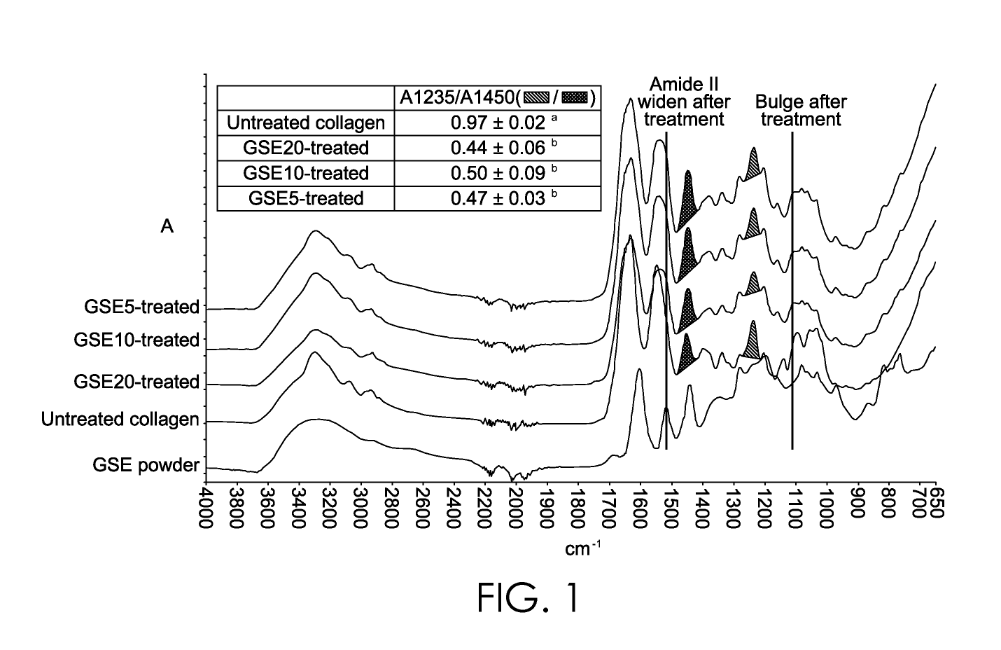

FIG. 1 shows the FTIR spectra of the dentin films treated with the GSE-containing etchant formulations, natural demineralized dentin collagen (“untreated collagen”), and GSE powder. The FTIR spectra for dentin films treated with the GA-containing etchant formulation did not cause any perceivable change compared to demineralized collagen's FTIR spectra (as shown in Liu, Y., Wang, Y., (2013) Proanthocyanidins' efficacy in stabilizing dentin collagen against enzymatic degradation: MALDI-TOF and FTIR analyses. Journal of Dentistry 41(6):535-542) and are therefore not shown. In contrast, all GSE-containing etchant formulation treatments resulted in pronounced alterations, including wider amide-II bands (˜1540 cm⁻¹), bulge formation (˜1108 cm⁻¹), and significantly-decreased amide-III (shade, ˜1235 cm⁻¹) to CH₂-bending (grid, ˜1450 cm⁻¹) ratio (A1235/A1450, FIG. 1A-inset). The A1235/A1450 ratios were equivalent within the group of dentin films treated with the GSE-containing etchant formulations regardless of the phosphoric acid concentrations tested. Following the GSE-containing etchant formulation treatments, the widening of amide II and formation of bulge are attributed to the spectral trace of GSE-powder, and so is the decreased amide-III/CH₂-bending (A1235/A1450) ratio (inset), because GSE-powder's spectrum augments collagen's CH₂-bending but contributes little to its amide. Statistical analysis was performed with Statistical Product and Service Solutions (SPSS, Version 21, IBM SPSS, Inc., Chicago, Ill.) and a nominal level of significance at 0.05. The normality of distribution and homogeneity of variances were confirmed with Shapiro-Wilk and Levene's tests, respectively. Comparison of means was performed with one-way ANOVA and Turkey's post hoc test. Values with the same superscripts (a and b in the inset to FIG. 1) indicate statistical equivalence (n=5).

The short treatment-time (30 seconds) and extensive rinsing (30 minutes) substantiated that demineralized dentin-collagen interacts with and immobilizes proanthocyanidins in the presence of 20%-5% phosphoric acid. Additionally, the A1235/A1450 ratio, listed in the FIG. 1 inset, indicates that the amount of proanthocyanidins incorporated into the collagen was not affected by the phosphoric acid concentration.

### Example 2: Proanthocyanidin-Rich Grape Seed Extract can Protect Demineralized Dentin from Collagenase Digestion in the Presence of Phosphoric Acid

The demineralized dentin films treated with the GA-containing or GSE-containing etchant formulations of Example 1 were also subjected to collagenase treatment. Particularly, the demineralized dentin films treated with the GA-containing or GSE-containing etchant formulations were incubated in 30 μl of the collagenase-containing solution at 37° C. for 1 hour, and the percentage of digested film in each sample supernatant was determined as follows.

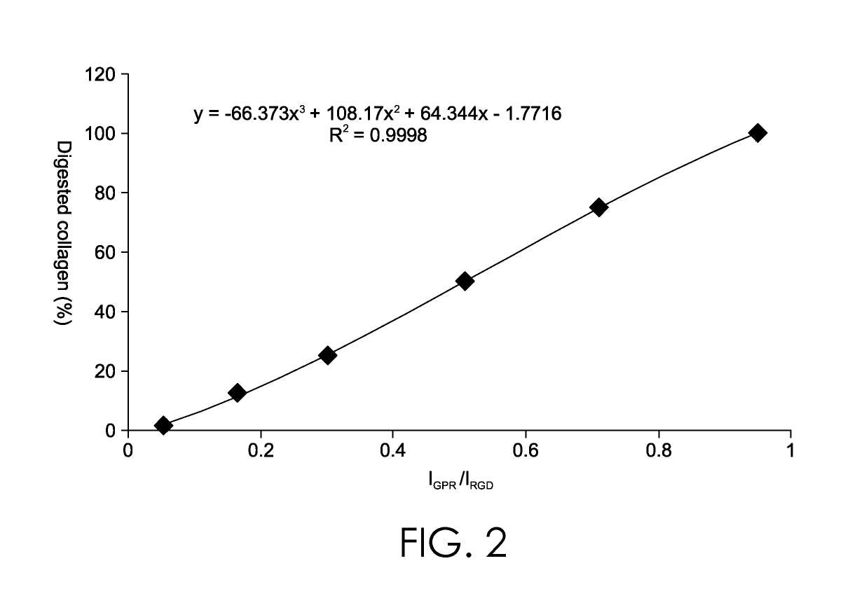

First, to obtain the standard curve, demineralized but untreated films were separately digested at 37° C. for 1 hour in 0.1% (w/v) collagenase solutions of various volumes: 30 μl, 40 μl, 60 μl, 120 μl, 240 μl and 1920 μl, respectively. Since all films were completely digested in 1 hour, this resulted in standard digests with the degree of collagen film degradation at 100%, 75%, 50%, 25%, 12.5% and 1.5% per 30 μl of collagenase, respectively. For each standard digest, 8 μl of the liquid was transferred to an Eppendorf vial, and 4 μl of Arg-Gly-Asp tripeptide solution (0.25 mg/ml) was added to the vial and thoroughly mixed with the digest liquid. A positive ion detection matrix was prepared by dissolving 2,5-dihydroxybenzoic acid (DHB) in acetonitrile/water (60/40, v/v) containing 0.2% trifluoroacetic acid to a final concentration of 20 mg/ml. After introducing the DHB matrix into the digest/tripeptide mixture at a 2:1 volume ratio, 1 μl of the resultant solution was spotted on the target for subsequent mass spectroscopic analysis using a Voyager DE Pro MALDI-TOF mass spectrometer (Applied Biosystems, Foster City, Calif., USA), operating in positive, reflector mode. For each MS spectrum, the peak intensities at mass-to-charge (m/z) ratios of 329.2 and 351.2 were summed (IGPR), representing the Gly-Pro-Arg tripeptide from digested collagen. The same was done for the peaks at 347.2 and 369.2 (IRGD), representing the Arg-Gly-Asp tripeptide internal standard. The intensity ratios (IGPR/IRGD) of the standard digests were plotted against their corresponding percentage of digested collagen film, and the standard curve was obtained by the least-square curve fitting using a third-order polynomial function. The standard curve is depicted in FIG. 2.

Next, the IGPR/IRGD ratios of sample supernatants were acquired in the same way, and the percentage of digested collagen film in each sample supernatant was calculated by inserting its respective IGPR/IRGD ratio into the standard function displayed in FIG. 2 and in Formula I below.

y=−66.373x³+108.17x²+64.344x−1.7716 (I)

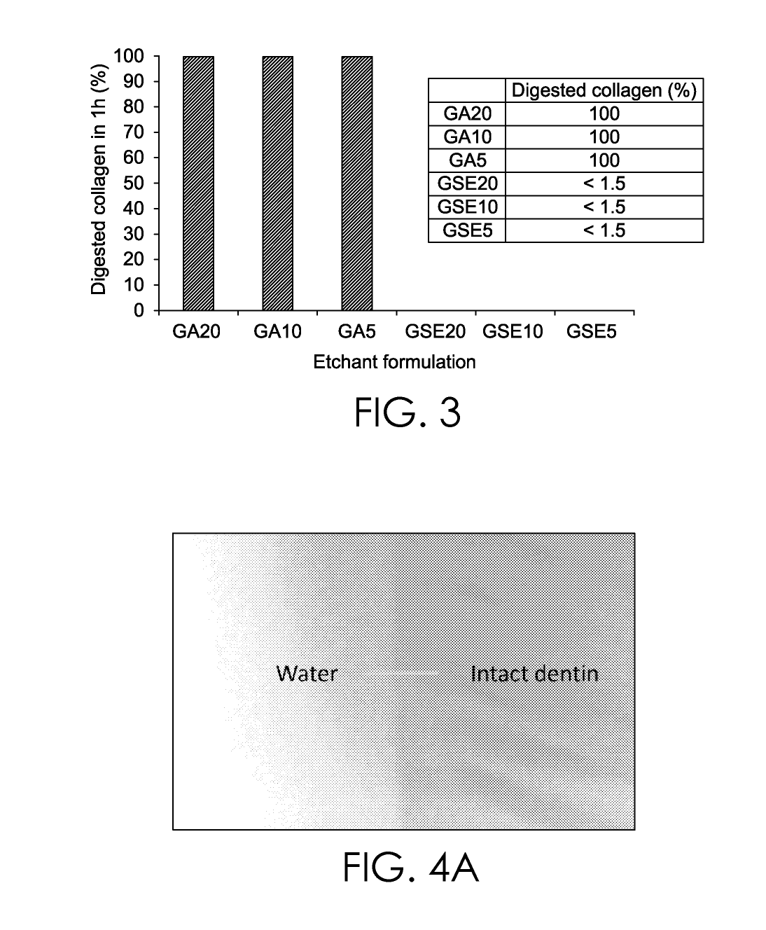

In case the IGPR/IRGD ratio of certain supernatant group was not statistically higher than that of standard digests containing 1.5% of digested collagen, which is the detection limit determined in a previous study (Liu Y, Wang Y (2013). Proanthocyanidins' efficacy in stabilizing dentin collagen against enzymatic degradation: MALDI-TOF and FTIR analyses. Journal of Dentistry 41(6):535-542), the percentage of digested dentin collagen film for that group was marked “<1.5%.” The results showing the percentage of dentin film degraded by 1 hour of bacterial collagenase digestion are illustrated in FIG. 3.

Results of the digestion assay shown in FIG. 3 matched well with the FTIR analysis with reference to FIG. 1. As seen in FIG. 3, the GA-containing dental etchant formulation-treated demineralized dentin collagen, which exhibited FTIR spectra unaltered from natural demineralized dentin collagen, was completely digested in 1 hour, like natural demineralized dentin collagen (data not shown). Conversely, the GSE-containing dental etchant formulation-treated DD-collagen, which had equivalent A1235/A1450 ratios, was invariably inert. Thus, these data demonstrate that the proanthocyanidin-rich GSE can protect demineralized dentin collagen from collagenase in the presence of phosphoric acid (e.g., a low pH environment), unlike glutaraldehyde.

### Example 3: Evaluation of the Etching Performance of GSE-Containing Dental Etchant Formulations

Ten molars were processed into dentin slabs as follows. After crown removal, a uniform smear layer was created on the dentin surface utilizing wet 600 grit Silicone Carbide (SiC) sandpaper (Buehler) for 30 seconds. Further sections were made in the occlusal-apical direction at 1 mm (for micro-Raman and SEM) or 0.5 mm increments (for TEM), followed by one cut parallel to, and ˜1.5 mm below, the abraded surface to free the slabs. Slabs for micro-Raman and SEM were notched at the mid-position from the side opposite to the abraded surface for the purpose of subsequent fracturing. A total of 42 notched (18 for micro-Raman, 24 for SEM) and 12 un-notched slabs (for TEM) were prepared.

For micro-Raman, the abraded surfaces of slabs were etched with the GSE20, GSE10, and GSE5 formulations (n=6) for 30 seconds. After 30 minutes of rinsing, the slabs were cryo-fractured in liquid nitrogen, and placed under focus of a 100× water-immersion lens of LabRam HR800 Raman spectrometer (Horiba Jobin Yvon, Edison, N.J.) with monochromatic He—Ne laser (operating at 632.8 nm). Spectra were acquired over the region of 500-1800 cm⁻¹, with a 30 second acquisition time and at positions corresponding to 1 μm intervals across the interfaces of water, demineralized dentin (DD), and intact dentin (ID). Each specimen was scanned three times at randomly-selected locations, and the distance between water-DD and DD-ID interfaces in each scan was averaged to one value, representing the etching-depth for the specimen.

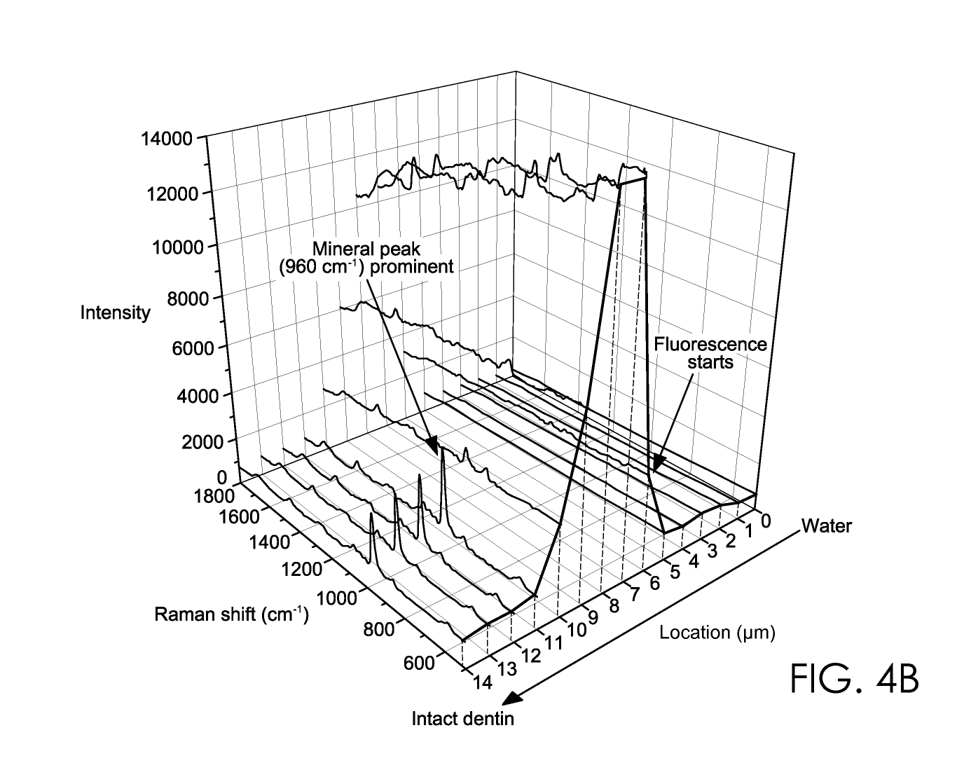

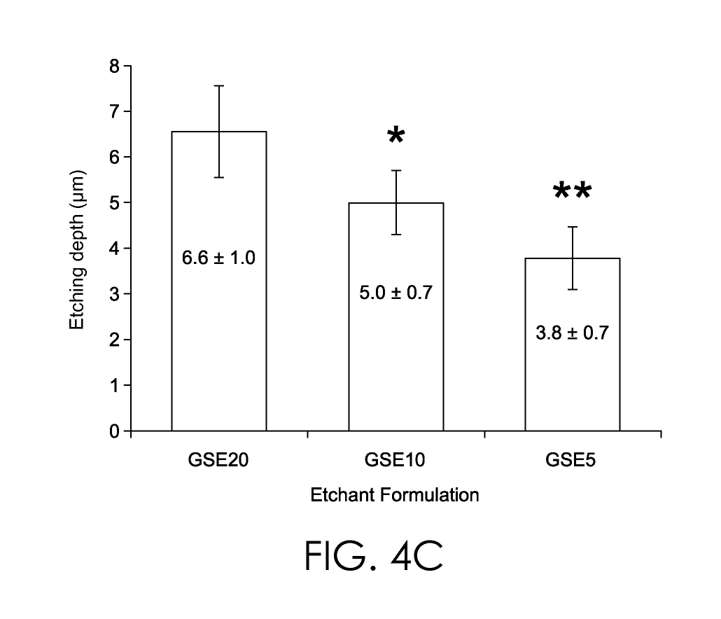

A representative optical image of a scanned interface is shown in FIG. 4A. Particularly, an optical image of the fractured cross-section of a representative specimen etched by GSE10 for 30 seconds is shown in FIG. 4A.

FIG. 4B depicts the Raman spectra collected from the specimen shown in FIG. 4A. As spectra collection started from water, flat, low-intensity lines were recorded (see at 0-5 μm). Owing to GSE's fluorescence, no well-defined Raman spectra could be acquired when scanning into DD, but the abruptly-elevated baseline (see at 6 μm) indicated the start of fluorescence and therefore the water-DD interface. The depth of the water-DD interface also can indicate the etching depth. Moving closer to ID, the interference of fluorescence faded, and the DD-ID interface was marked by the emergence of spectrum with prominent mineral peak at 960 cm⁻¹ (see at 11 μm).

FIG. 4C depicts the 30 second etching depth of the GSE20, GSE10, and GSE5 treated dentin slabs as determined based on the Raman spectra. Statistical analysis was performed with Statistical Product and Service Solutions (SPSS, Version 21, IBM SPSS, Inc., Chicago, Ill.) and a nominal level of significance at 0.05. The normality of distribution and homogeneity of variances were confirmed with Shapiro-Wilk and Levene's tests, respectively. Comparison of means was performed with one-way ANOVA and Turkey's post hoc test. Statistically different values are indicated by * and ** (n=6).

### Example 4: GSE-Containing Etchant Formulations' Ability to Stabilize Collagen while Etching Dentin

Slabs for SEM were etched for 30 seconds on the abraded surfaces with one of the six GA- or GSE-containing etchant formulations discussed above with reference to Example 1, rinsed for 30 minutes, and then underwent 1 hour of collagenase-digestion or DW-immersion at 37° C. Specimens for each etching-digestion combination were prepared in duplicates. Slabs for TEM were etched, rinsed and digested in the same way as SEM specimens (also in duplicates) except that only the GSE-containing etchant formulations were investigated. For both SEM and TEM, the subsequent specimen processing (including fracturing of SEM specimens) and observation followed the protocol described in Liu Y, Dusevich V, Wang Y (2013). Proanthocyanidins rapidly stabilize the demineralized dentin layer. Journal of Dental Research 92(8):746-752, hereby incorporated in its entirety.

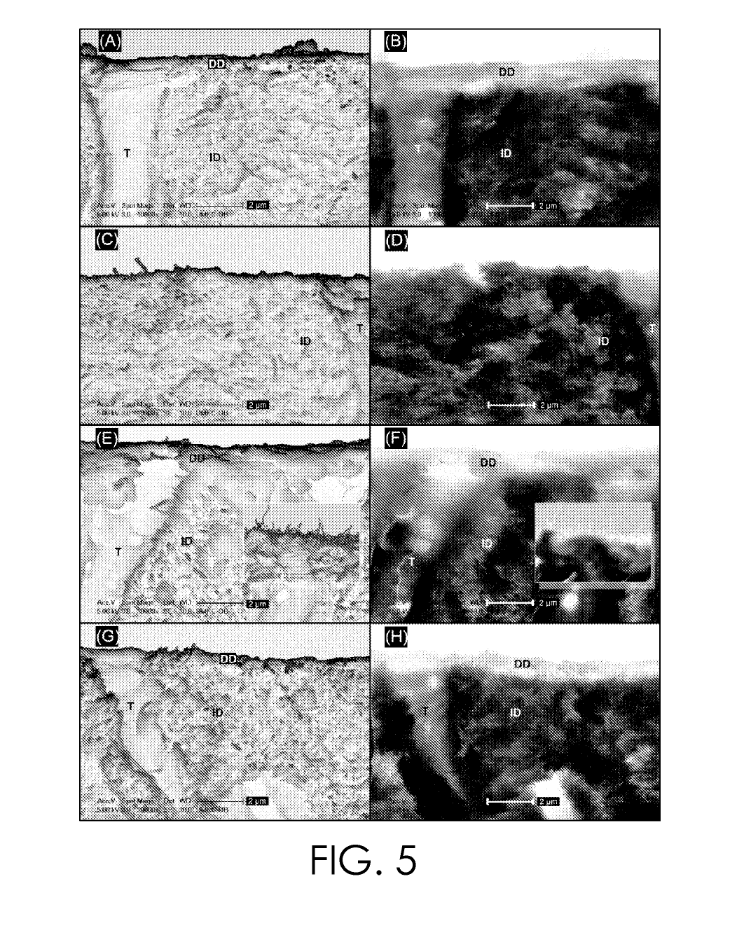

FIGS. 5A, 5C, 5E, and 5G show the SEM images of cross-sections of fractured dentin slabs, and FIGS. 5B, 5D, 5F, and 5H, respectively, show the back-scattered electron SEM images of the same location (DD=demineralized dentin; ID=intact dentin; T=tubule). FIGS. 5A and 5B show the typical morphology of etched dentin pre-digestion, regardless of the presence of GA or GSE. FIGS. 5C and 5D show dentin, etched with the GA-containing etchant formulation after 1 hour of digestion, showing no demineralized layer left. FIGS. 5E and 5F show dentin, etched with the GSE20 formulation, after 1 hour of digestion, featuring a thin demineralized film “draping” over the edge, and “hair-like” morphology on the complementary piece (insets). FIGS. 5G and 5H show dentin, etched with the GSE5 formulation, after 1 hour of digestion, showing a morphology unchanged from pre-digestion (FIGS. 5A and 5B). The dentin etched with the GSE10 formulation is not shown, as it showed similar morphology to that etched with the GSE5 formulation as shown in FIGS. 5G and 5H.

When not challenged by collagenase digestion, the cross-section of etched dentin shared similar morphological traits regardless of the presence of GA or GSE (see FIGS. 5A and 5B). These SEM images of the pre-digested dentin revealed distinct collagen fibrils seen under secondary-electron-SEM (FIG. 5A) in the DD-layer characterized by dark color under back-scattered electron SEM (FIG. 5B). After digestion, the DD layer of the GA-containing etchant formulation-treated specimen was completely gone (FIGS. 5C and 5D). In contrast, the DD layer of the samples treated with GSE-containing etchant formulations were still present, but its appearance depended upon phosphoric acid concentration. The GSE20-etched specimen had a thin, featureless demineralized film draping over the edge (FIGS. 5E and 5F), and the film seemed to be torn-off from the complementary piece, leaving “hair-like” DD fibrils on it (FIGS. 5E and 5F insets). In comparison, the DD-layers of GSE5-etched dentin were not altered by the collagenase digestion, as shown in FIGS. 5G and 5H (the DD-layers of GSE10-etched dentin showed similar morphology to that shown in FIGS. 5G and 5H and are not shown).

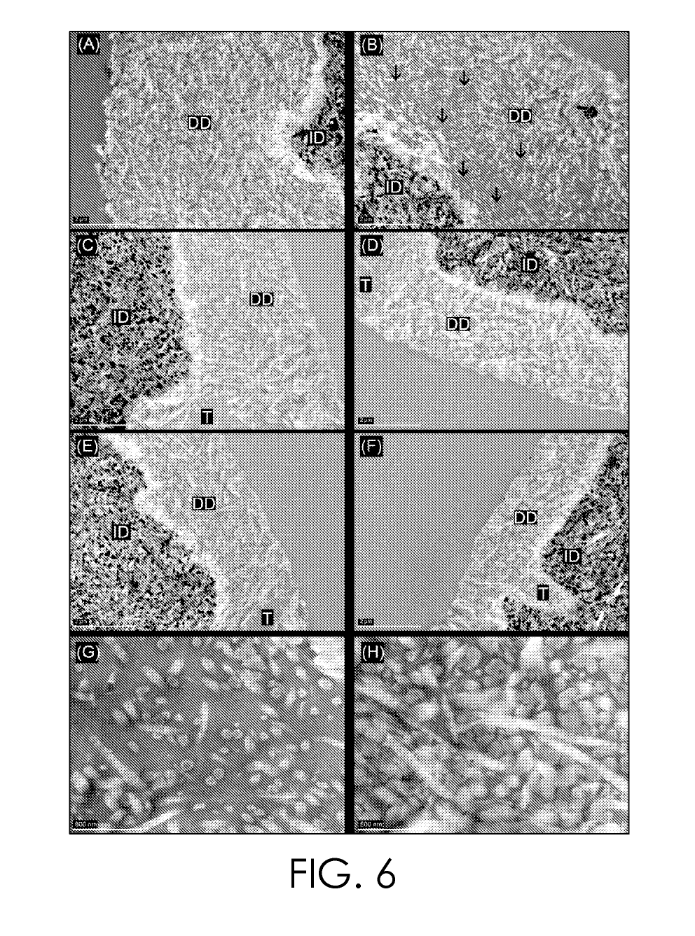

FIGS. 6A, 6C, and 6E show TEM images of dentin slab specimens etched with GSE20, GSE10, and GSE5 etchant formulations, respectively, without collagenase digestion. FIGS. 6B, 6D, and 6F show TEM images of dentin slab specimens etched with GSE20, GSE10, and GSE5 etchant formulations, respectively, after 1 hour of collagenase digestion. FIGS. 6G and 6H show high magnification views of the DD layer shown in FIG. 6B near the arrows and finger, respectively.

Consistent with the SEM data shown in FIGS. 5A-5H, the TEM images of undigested, specimens etched with GSE-containing etchant formulations (GSE20, GSE10, and GSE5), regardless of the phosphoric acid concentration, displayed a DD-layer (FIGS. 6A, 6C, and 6E) in which fibrils exhibit the characteristic 67-nm banding pattern of type-I collagen (see FIG. 5H). Additionally, the DD layers of the GSE20, GSE10, and GSE5 treated specimens exhibited densely-packed collagen fibrils pre-digestion (FIGS. 6A, 6C, and 6E). After digestion, the DD in the GSE20 etched specimen was no longer intact, as voids were evident (FIG. 6B, arrows) close to the bottom of DD layer. Collagen fibrils in the voids were much scarcer (FIG. 6G) than intact DD (FIG. 6H). In comparison, the GSE10 and GSE5 etched specimens had no voids across the entire DD layer (FIGS. 6D, 6F) post-digestion.

The top DD-layer of GSE20-etched specimen may be resistant to bacterial collagenase, but the bottom-portion may be substantially degraded (see FIG. 6B). It thus, may have resulted in the SEM specimen of GSE20-etched dentin to have a collagen film floating on weak collagen-fibril support after digestion and before fracturing (see FIGS. 5E and 5F insets). When fractured, cracks propagated along the weak collagen-fibril support then through the collagen film, hence producing the “drape-like” appearance on one piece and “hair-like” appearance on the complementary piece (see FIGS. 5E and 5F insets).

Without being bound by any particular theory, this collagen degradation in the GSE20-etched DD-layer (FIG. 6B) may not be due to higher phosphoric acid concentration, e.g., see the collagenase protection exhibited with this formulation in FIG. 3 and Example 2. Therefore, without by being bound by any particular theory, the collagen degradation in the GSE-20 etched layer may exemplify an unsynchronized penetration of phosphoric acid and one or more compounds in the GSE, as GSE may be comprised of (epi)catechin monomers and oligomers, which are not only intrinsically more hydrophobic and less mobile than the small, hydrophilic phosphoric acid, but may self-assemble into even bigger micelles in hydro-alcoholic media at acidic pH. Thus, without being bound by any particular theory, GSE may diffuse slower than phosphoric within the intra-tubular space which may be filled with water (and/or the retention of collagen may have retarded the diffusion of GSE and/or GSE-micelles in the inter-tubular space as well), and thus, the phosphoric acid in the GSE20 etchant may have etched too fast for GSE to catch up, which may leave the collagen fibrils at the etching-front not fully-protected at the top of DD-layer.

From the foregoing, it will be seen that this disclosure is one well adapted to attain all the ends and objects hereinabove set forth together with other advantages which are obvious and which are inherent to the structure.

It will be understood that certain features and subcombinations are of utility and may be employed without reference to other features and subcombinations. This is contemplated by and is within the scope of the claims.

Since many possible aspects may be made of the disclosure herein without departing from the scope thereof, it is to be understood that all matter herein set forth or shown in the accompanying drawings is to be interpreted as illustrative and not in a limiting sense.

What is claimed is:

1. A dental etchant composition comprising: at least one acid; and one or more dentin collagen cross-linking agents.

2. The dental etchant composition according to claim 1, wherein the one or more dentin collagen cross-linking agents comprise one or more polyphenols.

3. The dental etchant composition according to claim 2, wherein the one or more polyphenols comprise one or more proanthocyanidins.

4. The dental etchant composition according to claim 1, wherein the at least one acid comprises phosphoric acid, and wherein the phosphoric acid is present in an amount of at least about 1 wt. % and no more than about 40 wt. %.

5. The dental etchant composition according to claim 1, wherein the one or more dentin collagen cross-linking agents are present in at least one of a plant extract or a plant seed extract, and wherein the at least one of a plant extract or a plant seed extract is present in an amount of at least about 0.1 wt. % to no more than about 15 wt. %.

6. The dental etchant composition according to claim 1, wherein a pH of the dental etchant composition is less than about 2.

7. The dental etchant composition according to claim 1, further comprising one or more rheology modifiers selected from the group consisting of: silica, polyvinyl alcohol, and a combination thereof.

8. A process for the production of a dental etchant composition, the process comprising: combining at least one acid with one or more dentin collagen cross-linking agents to form a dental etchant composition.

9. The process according to claim 8, wherein the one or more dentin collagen cross-linking agents comprise one or more polyphenols.

10. The process according to claim 9, wherein the one or more polyphenols comprise one or more proanthocyanidins.

11. The process according to claim 8, wherein the dental etchant composition has a pH of less than about 2.

12. The process according to claim 8, wherein the at least one acid comprises phosphoric acid, maleic acid, citric acid, or a combination thereof.

13. The process according to claim 8, wherein the one or more dentin collagen cross-linking agents are present in at least one of a plant extract or a plant seed extract, and wherein the at least one of a plant extract or a plant seed extract is present in an amount of at least about 0.1 wt. % to no more than about 15 wt. %.

14. A method for performing a dental restoration on a mammal's tooth, the method comprising: contacting at least a portion of a mammal's tooth with a dental etchant composition, the dental etchant composition comprising: one or more acids and one or more dentin collagen cross-linking agents; and removing at least a portion of the dental etchant composition from the mammal's tooth.

15. The method according to claim 14, wherein the dental etchant composition is present on the mammal's tooth for at least about 15 seconds.

16. The method according to claim 14, wherein the one or more dentin collagen cross-linking agents comprise one or more polyphenols.

17. The method according claim 14, wherein the one or more acids comprise phosphoric acid, maleic acid, citric acid, or a combination thereof.

18. The method according to claim 17, wherein the one or more acids comprise phosphoric acid in an amount of at least about 1 wt. % to no more than about 40 wt. %.

19. The method according to claim 14, wherein the one or more dentin collagen cross-linking agents are present in at least one of a plant extract or a plant seed extract.

20. The method according to claim 19 wherein the at least one of a plant extract or a plant seed extract comprises grape seed extract, wherein the grape seed extract is present in the dental etchant composition in an amount of at least about 0.1 wt. % to no more than about 15 wt. %.
